(19) United States
(12) Patent Application Publication
(10) Pub. No.: US 2005/0261723 A1
(43) Pub. Date: Nov. 24, 2005

(54) Folded balloon for catheter

(75) Inventors: Justin Goshgarian, Santa Rosa, CA (US); Aaron J. Miller, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/134,699

(22) Filed: May 20, 2005

Related U.S. Application Data
(60) Provisional application No. 60/572,757, filed on May 21, 2004.

(51) Int. Cl. A61M 29/00

(52) U.S. Cl. 606/192

(57) ABSTRACT

A balloon assembly with an inner member and a balloon mounted thereon and folded in an accordion manner. The balloon comprises a plurality of pleats on either side of the inner member. The balloon can have an equal number of pleats on either side of the first inner member, or can be folded in an asymmetrical manner. The balloon can also be folded in a tri-fold accordion manner. Each of the pleats comprising an ascending fold and a descending fold, the ascending and descending folds connected by an apex. The pleats can also be connected by an apex. Alternatively, the ascending and descending folds can be connected by a curved portion, and the pleats can also be connected by a curved portion. The balloon assembly can also be a dual balloon assembly, with the second balloon mounted on a second inner member and also folded in an accordion manner.

### RELATED APPLICATIONS

This application claims the benefit of U.S. Provisional Patent Application 60/572,757 filed May 21, 2004.

### BACKGROUND OF THE INVENTION

1. Field of the Invention

The present invention is related to balloons, and more particularly, to folded balloons for use with low profile balloon catheter systems.

2. Background Art

Balloon catheters are used to treat coronary disease. In one common application, a balloon catheter is used in a percutaneous transluminal coronary angioplasty (PTCA) procedure. In a PTCA procedure, the balloon catheter is threaded through an artery to a site of a lesion. The balloon is inflated to compress plaque associated with coronary artery disease against the artery's walls. This creates a larger opening in the artery and helps restore adequate blood flow. The balloon is then deflated and withdrawn from the vessel.

Balloon catheters are also used in combination with stents to treat coronary disease. A stent is a miniature mesh tube, typically made from steel. The balloon catheter and stent are threaded through an artery to the site of a lesion. The balloon is inflated to expand the stent. The stent is flexible, yet strong enough to remain in place after the balloon is deflated and cleared. Once the stent is in place, the balloon is deflated and withdrawn from the vessel.

Upon deflation, the balloon tends to re-form to a folded, wrapped state due to its shape memory. In some cases, pressures in the vessel can cause the number of folds that re-form upon deflation to be different from the number of initial folds in the balloon. Larger diameter balloons with higher numbers of folds are especially more likely to re-form to a lesser number of folds upon deflation.

FIG. 1 shows a cross-section of a distal portion of a conventional multifold (spiral fold) balloon stent delivery catheter 140 that includes a stent 120 and a multifold balloon angioplasty catheter 150. Near a distal end of the multifold balloon angioplasty catheter 150, an angioplasty balloon 152 with folds 152A, 152B, 152C, 152D and 152E is attached to an inner shaft (inner member) 112 which has a guide wire 130. The inner member 112 is in fluid communication with the balloon 152, and the inner member 112 is used to inflate and deflate the balloon 152 so as to expand the stent 120 into a wall of a vessel of a human body (not shown). A radiopaque marker band (not shown in the figures) fixed to the inner member 112 provides a reference for identifying the position of the balloon 152 and stent 120. During expansion of the balloon 152, the folds 152A, 152B, 152C, 152D and 152E exert frictional radial forces against the inside surface of the stent 120.

In some cases where a bifurcation in the vessel is present, a dual balloon catheter may be necessary. In FIG. 2, a conventional dual balloon catheter assembly 220 is shown, which includes a pair of balloon catheters 222 and 224. Each balloon catheter includes a shaft (inner member) 226 and 228, and balloons 230 and 232. The balloons on each balloon catheter 226 and 228 are folded in same manner as described above with reference to FIG. 1 such that each catheter has a circular cross-section. At least a portion of the catheters 222 and 224 may be contained within a guide catheter 234 that keeps the inner members 226 and 228 in close proximity to one another, thereby providing the assembly 220 with a relatively compact profile. The guide catheter 234 may be configured to contain the catheters 222 and 224 in the substantially circular lumen 235.

The assembly 220 may be configured to have a circular cross-sectional profile throughout its entire length or a portion thereof. In the embodiment shown in FIG. 2, the guide catheter 234 is disposed about the catheters 222 and 224 and provides the assembly 220 with a substantially circular cross-section. However, due to the circular cross-section of each of the catheters 222 and 224, a significant portion of the lumen 235 is wasted space 239, giving the overall structure a large crossing profile.

Accordingly, there is a need in the art for a novel approach to folding balloons that permits reduction of the crossing profile of dual balloon catheter assemblies.

### SUMMARY OF THE INVENTION

The present invention is directed to a folded balloon for catheter applications that substantially obviates one or more of the problems and disadvantages of the related art.

An embodiment of the present invention includes a dual balloon assembly with a first inner member and a first balloon mounted thereon and folded in an accordion manner. A second inner member has a second balloon mounted thereon. The first balloon has a plurality of pleats on each side of the first inner member. The first balloon can have an equal number of pleats on either side of the first inner member, or can be folded in an asymmetrical manner. The first balloon can also be folded in a tri-fold accordion manner. Each of the pleats includes an ascending fold and a descending fold connected to each other by an apex. The pleats can also be connected to each other by an apex. Alternatively, the ascending and descending folds can be connected to each other by curved portions, and the pleats can also be connected to each other by curved portions.

A stent can be mounted over each of the first and second balloons. A guide catheter can be disposed over the first and second balloons. The first balloon can be formed of nylon, polyethylene teraphthalate, polyethylene, polypropylene, polyvinyl chloride, and elastomer.

The second balloon can also be folded in the accordion manner to have a plurality of pleats on either side of the second inner member. The pleats of the first and second balloons can be oriented generally perpendicular to a line connecting centers of the first and second inner members.

In another embodiment of the invention, a balloon assembly includes an inner member and a balloon mounted on the inner member and folded in an accordion manner. The balloon includes a plurality of pleats on each side of the inner member. A stent can be mounted over the balloon, and a guide catheter can be disposed over the balloon.

In another embodiment of the invention, a balloon assembly includes a plurality of inner members and a plurality of accordion-folded balloons mounted on corresponding inner members.

In another embodiment of the invention, a method of folding a balloon includes attaching a balloon to an inner member; forming the balloon into an accordion, or batwing shape; and placing a sheath over the balloon. The forming step can include drawing a balloon through a mold that includes a plurality of ridges. The mold includes ridges that guide material of the balloon to form pleats of the accordion shape. An intravascular prosthesis, such as a stent or a stent-graft, can be placed over the balloon. The forming step forms a plurality of pleats. The pleats can be formed symmetrically or asymmetrically about the inner member. The forming step can also form the balloon into a tri-fold accordion shape. The forming step can form a plurality of pleats of varying size.

In another embodiment of the invention, a method of forming a balloon assembly includes attaching a plurality of balloons to corresponding inner members; forming each balloon into an accordion shape; and placing a sheath over each balloon.

In another embodiment of the invention, a method of delivering a stent includes the steps of inserting a catheter into a blood vessel, the catheter including a balloon mounted on an inner member of the catheter and a stent mounted on the balloon; inflating the balloon to deliver the stent; deflating the balloon; and withdrawing the catheter from the blood vessel. The balloon mounted on the inner member is folded in an accordion manner. The stent can be a bifurcated stent, or a non-bifurcated stent.

Additional features and advantages of the invention will be set forth in the description that follows, and in part will be apparent from the description, or may be learned by practice of the invention. The advantages of the invention will be realized and attained by the structure and particularly pointed out in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to illustrate exemplary embodiments of the invention and are incorporated in and constitute a part of this specification, illustrate embodiments of the invention and together with the description serve to explain the principles of the invention. In the drawings:

FIG. 1 shows a partial cross-section of a conventional balloon catheter.

FIG. 2 shows a conventional dual balloon catheter assembly.

FIG. 3 illustrates an embodiment of a folded balloon of the present invention.

FIG. 4 illustrates a side view of two balloons placed sequentially on an inner member and partly overlapping each other.

FIG. 5 shows a cross-sectional view of the folded balloon of FIG. 3.

FIG. 6 illustrates another embodiment of a folded balloon of the present invention, where pleats of the folded balloon have a variable size.

FIG. 7 illustrates another embodiment of the present invention with two side-by-side folded balloons.

FIG. 8 illustrates another embodiment of the present invention with asymmetrically folded balloons.

FIG. 9 illustrates another embodiment of the present invention using a tri-fold accordion balloon.

FIG. 10 illustrates a dual balloon arrangement that uses two tri-fold accordion balloons as shown in FIG. 9.

FIG. 11 illustrates yet another embodiment of the present invention that uses an arrangement of three tri-folded accordion balloons.

FIG. 12 illustrates yet another embodiment of a folded balloon of the present invention, where for every ascending fold on one side there is a corresponding ascending fold on the other side.

FIG. 13 illustrates yet another embodiment of a folded balloon of the present invention, with rounded portions used to connect the folds.

FIG. 14 illustrates an isometric view and FIG. 15 illustrates a side view of a mold used to fold a balloon of the present invention.

FIG. 16 illustrates a cross sectional view of upper and lower portions of the mold of FIG. 14, and FIG. 17 illustrates an isometric view of the lower portion of the mold of FIG. 14.

FIG. 18 illustrates a cross sectional view of an embodiment of a folded balloon of the present invention with a stent mounted thereon.

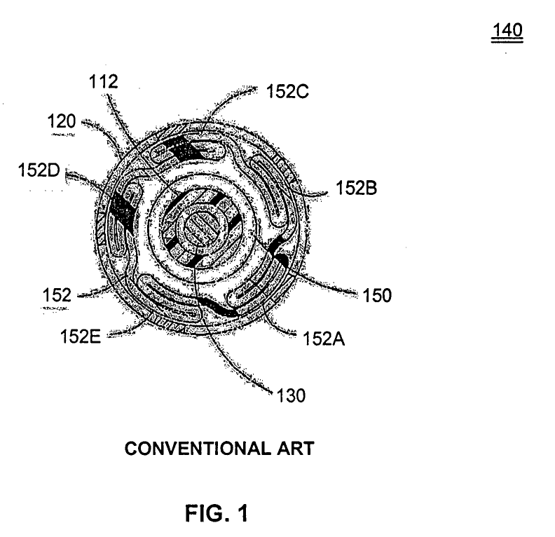

### DETAILED DESCRIPTION OF EMBODIMENTS OF THE INVENTION

Reference will now be made in detail to the embodiments of the present invention, examples of which are illustrated in the accompanying drawings.

It will be appreciated that in the discussion below, the sense of direction (up, down, left, right) is purely arbitrary and is used for illustrative purposes only to help explain the drawings.

During manufacture of the balloon catheter assembly, a balloon is bonded to an inner member of a catheter. The invention is also applicable to balloons that are attached to a guide catheter, as well as any type of intravascular catheters, such as, e.g., over-the-wire catheters. A series of processing steps are then carried out on the balloon. These balloon processing steps include folding and then baking the balloon to heat set in the folded and wrapped shape. Inspection and sterilization operations are also carried out on the balloon. After this processing, the balloon is covered with a protective sleeve and shipped.

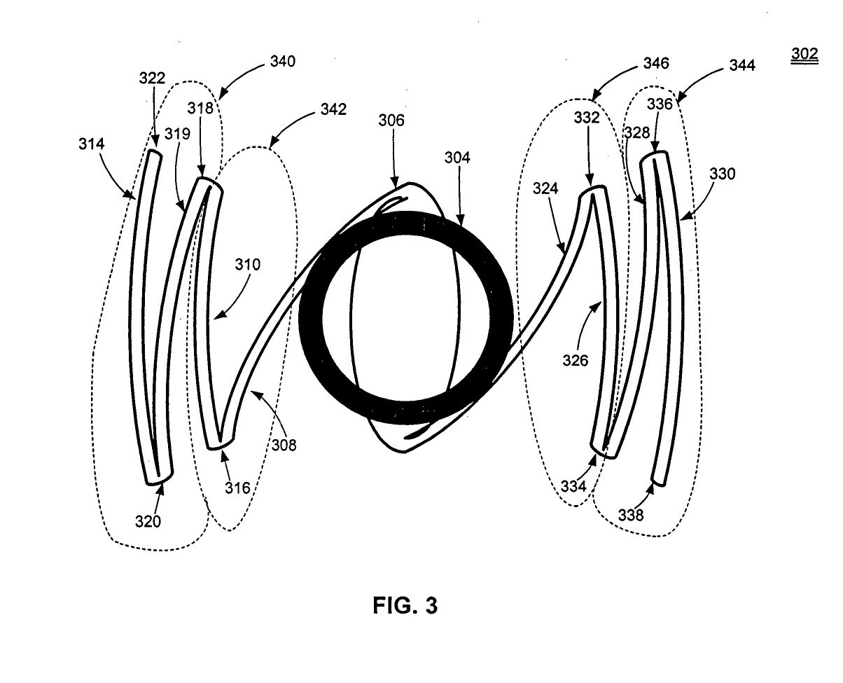

FIG. 3 illustrates an embodiment of a folded balloon of the present invention. As shown in FIG. 3, a balloon assembly 302 includes an inner member 304, which is typically 0.024 inches in diameter and can vary between 0.020 and 0.040 inches in diameter. Mounted on the inner member 304 is a balloon 306 that is shown in FIG. 3 in its folded state. The folded state shown in FIG. 3 may be described as a batwing, or an accordion-type folding. As further shown in FIG. 3, the balloon 306 is folded to include a number of pleats, designated by 340, 342, 344 and 346 in FIG. 3. Pleat 342 includes an ascending fold 308, an apex 316, and an ascending fold 310. Pleat 340 is formed by a descending fold 319, an apex 320, an ascending fold 314, and a termination at point 322.

Similarly, on the right side of FIG. 3, the balloon 306 is folded to have an ascending fold 324, an apex 332, a descending fold 326 (forming the pleat 346), an apex 334, an ascending fold 328, an apex 336, a descending fold 330, and an end or termination point 338 (forming the pleat 344).

It will be appreciated that although in FIG. 3, the balloon 306 is folded to have two pleats on the right, and two pleats on the left, more or fewer pleats may be used, depending on the size of the balloon 306 and the size of the inner member 304. For example, typical balloons for coronary applications are about 2 to 5 mm in diameter, when expanded. Balloons intended for some peripheral applications may be larger, as much as 10 to 12 mm in diameter, when expanded. It will be appreciated that larger balloons will usually, though not always, use more pleats for folding the balloon 306 to form the accordion arrangement illustrated in FIG. 3.

Balloon 306 is usually formed of a thin pliable material capable of expanding from a compact, collapsed state to an expanded diameter. Balloon 306 may be formed from polyethylene teraphthalate (PET) using a drawing and blow molding process so as to provide biaxial orientation to the material. PET balloons exhibit the desirable properties of high burst strength and relatively low radial expansion when inflated to high pressures. Alternatively, balloon 306 may be formed from nylon, polyethylene, polypropylene, polyvinyl chloride, elastomer or other material, as would be apparent to one skilled in the relevant art. Balloon 306 is typically approximately 2-4 cm long and is attachable to a distal portion of a catheter by methods known in the art, including gluing, melting or welding.

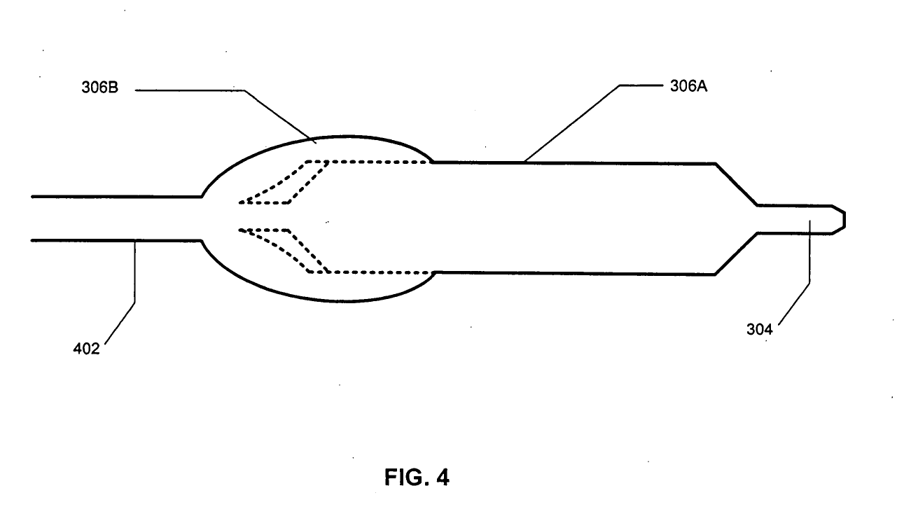

FIG. 4 illustrates a side view of two balloons 306A, 306B placed sequentially and partly overlapping on the inner member 304 of a catheter 402. If such a double balloon were to be folded in a conventional manner, it would have a relatively large crossing profile. However, if folded into the accordion shapes, the crossing profile could be made relatively small.

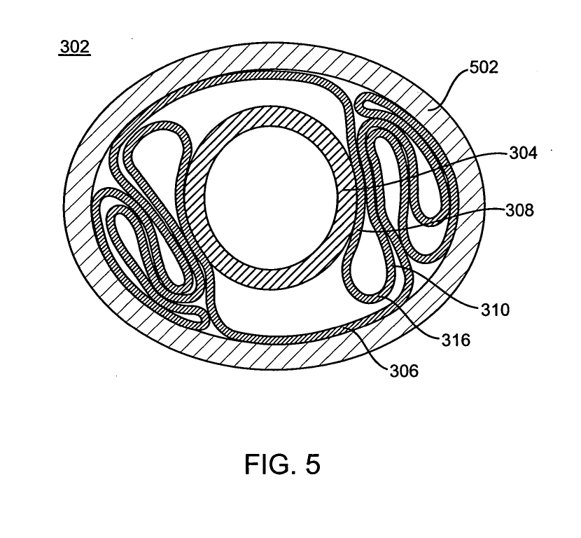

FIG. 5 shows a photograph of a cross-sectional view of a single balloon 306 that is folded into the accordion shape, and is placed within a sheath 502 after folding.

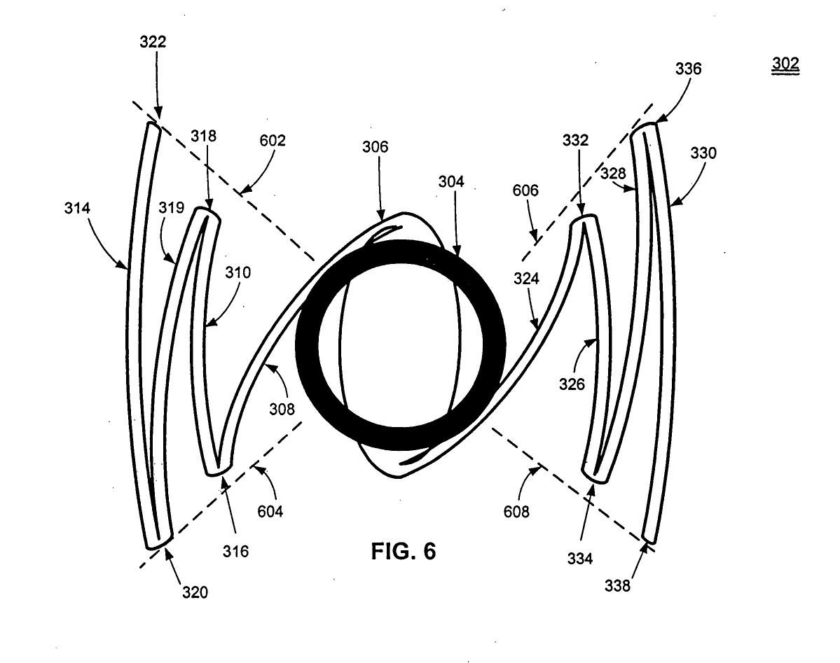

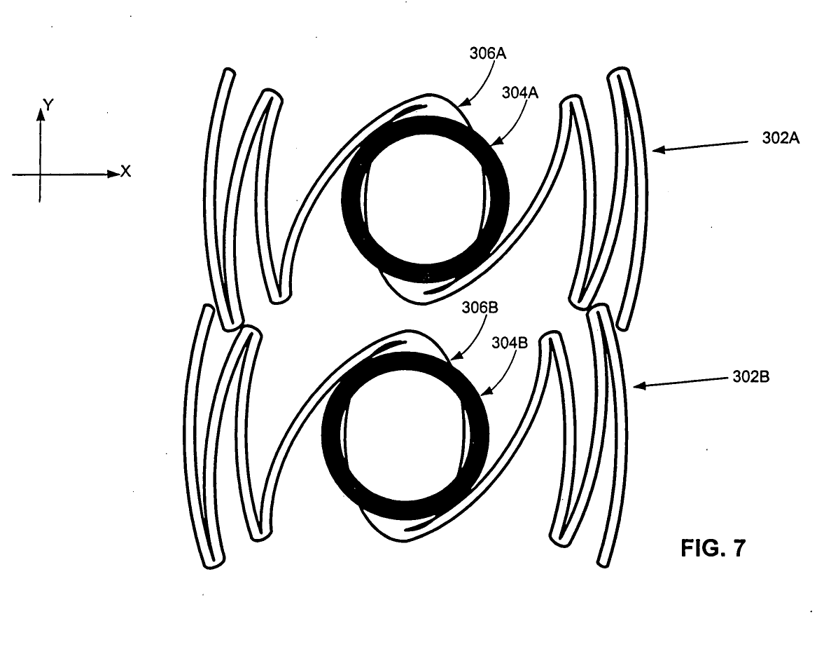

Although the embodiment shown in FIG. 5 uses pleats are roughly comparable in dimension to the diameter of the inner member 304 (see FIG. 3), this need not necessarily be the case. For example, FIG. 6 illustrates an alternative embodiment, where the pleats have a variable size, in this case, the outward pleats are larger than the inner pleats. Dashed lines 602, 604, 606 and 608 are added for illustrative purposes, to better illustrate that the outer folds are longer than the inner folds. Additionally, it is possible to have outer folds that are smaller than the inner folds (not shown in the figures). This may be advantageous when the size of the inner member 304 is relatively large. In that case, it is possible to use outer folds that are smaller than the inner folds, or folds that gradually decrease in length as one moves radially outward (left and right in FIG. 6) from the inner member 304.

FIG. 7 illustrates another embodiment of the present invention where two side-by-side folded balloons 302A, 302B are used. This figure helps illustrate how the overall crossing profile of the dual balloon/catheter arrangement can be reduced. The Y dimension in FIG. 7 is substantially comparable to the dimension of the two inner members 304A, 304B added together. When it comes to reducing the crossing profile of such a dual balloon/catheter assembly, it is typically the Y dimension that causes the primary difficulty. Note that although two distinct balloons 306A, 306B are shown in FIG. 7, it is also possible to use a single (large) balloon folded into the dual accordion shape shown in FIG. 7. Furthermore, the folded balloon of the present invention may be used side-by-side with a conventional spiral folded balloon.

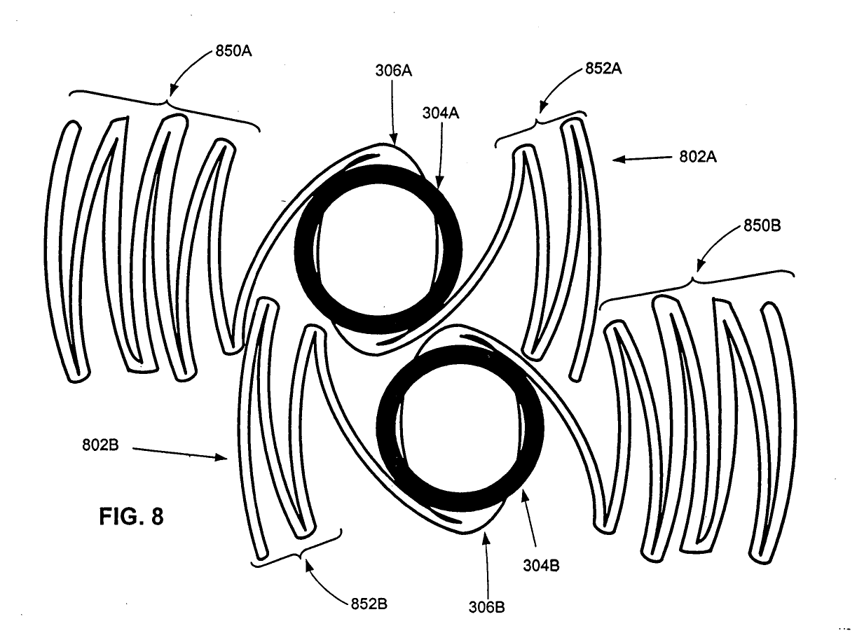

FIG. 8 illustrates another alternative embodiment of the present invention. In the embodiments shown in, for example, FIGS. 3, 6 and 7, the folding is symmetrical about the inner member 304. In other words, the same number of pleats is used on both the left and the right side of the inner member 304 in the embodiments shown in FIGS. 3, 6 and 7. However, this need not be the case. As shown in FIG. 8, two balloons 306A, 306B are mounted on inner members 304A, 304B. However, the number of pleats 850A on the left of the inner member 304A is different from the number of pleats 852A on the right of the inner member 304A. Similarly, the number of pleats 850B on the right of the inner number 304B is different than a number of pleats 852B on the left of the inner member 304B. This may be particularly advantageous for acentric balloons, and may allow further reduction in the crossing profile. It will also be appreciated that the two balloon assemblies 802A and 802B need not be the mirror images of each other. For example, it is possible to fold one of the two balloons symmetrically, as shown, for example, in FIG. 3, and to fold the other balloon asymmetrically.

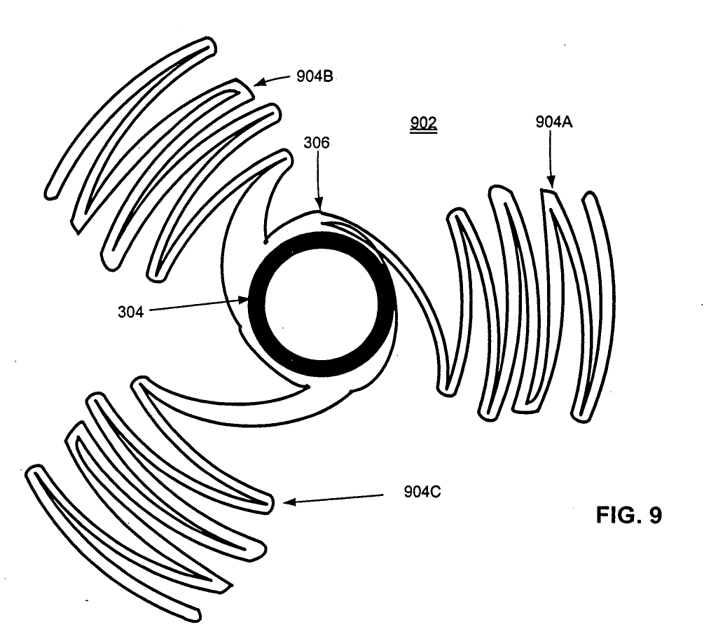

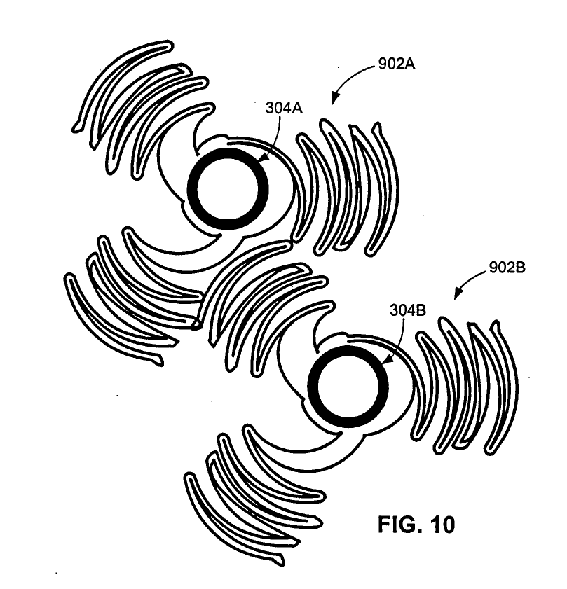

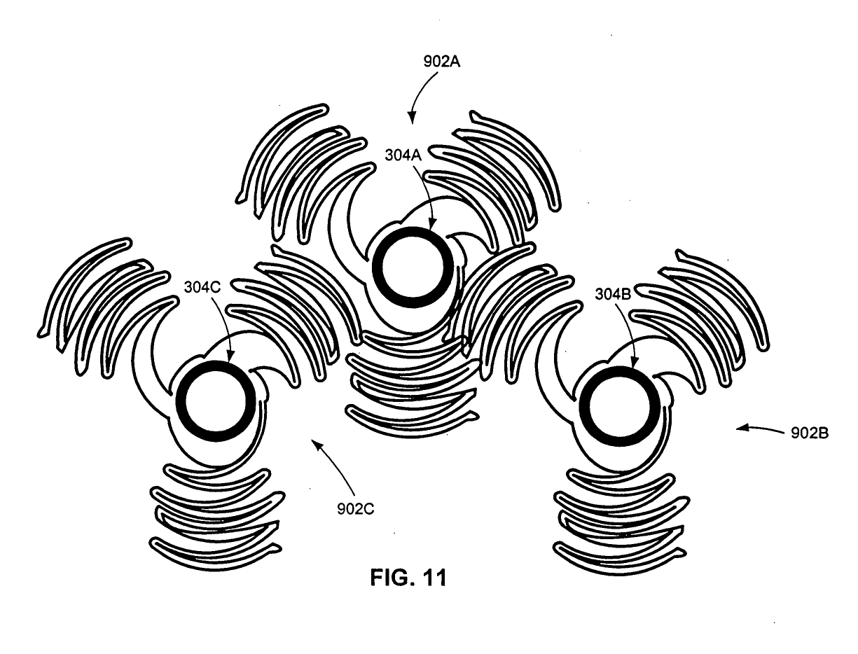

FIG. 9 illustrates yet another alternative embodiment of the present invention. As shown in FIG. 9, instead of a folding arrangement with two folded areas, the arrangement 902 of FIG. 9 may be thought of as a tri-fold, or as a Y-fold. The “arms” of the Y, viz, 904A, 904B, 904C come together at the inner member 304. FIG. 10 illustrates a dual balloon arrangement that uses the tri-fold illustrated in FIG. 9. As shown in FIG. 10, two tri-fold balloon assemblies 902A, 902B are arranged side by side.

FIG. 11 illustrates yet another embodiment of the present invention that uses three tri-folded balloon assemblies 902A, 902B, 902C arranged side-by-side. The inner members 304A, 304B, 304C form a roughly isosceles triangle, with the tri-fold balloons 902A-C arranged as shown in the figure, to reduce the crossing profile.

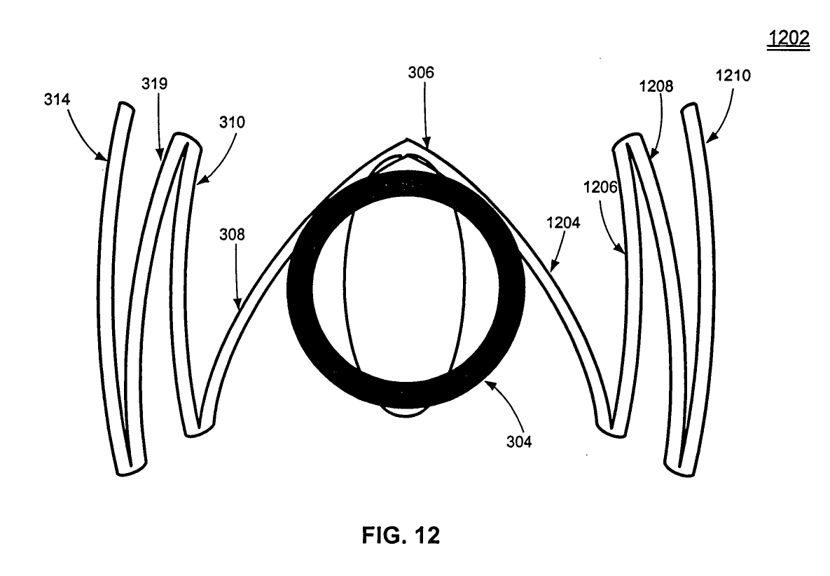

FIG. 12 illustrates yet another alternative embodiment of the present invention. In FIG. 3, described above, the pleats 340, 342 on the left of the inner member 304 are mirror images of the pleats 344, 346 on the right of the inner member 304. In other words, for every ascending fold on the left of the inner member 304, there is a corresponding descending member on the right of the inner member 304. In the balloon assembly 1202 shown in FIG. 12, for every ascending fold on the left there is a corresponding ascending fold on the right (i.e., folds 1204, 1206, 1208 and 1210).

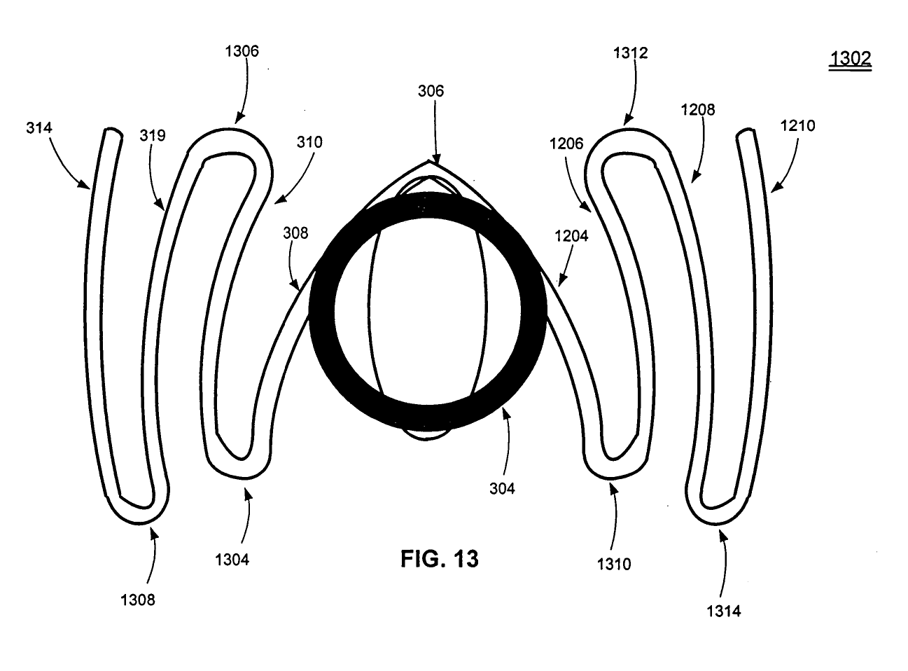

FIG. 13 illustrates yet another alternative embodiment of the present invention. In the embodiments described above, the apices (316, 318, 320, 332, 334, 336) of the balloon assembly 1302 are shown as being relatively “sharp”, or folded “tightly”. Alternatively, as shown in FIG. 13, rounded portions 1304, 1306, 1308, 1310, 1312, 1314 of the balloon assembly 1302 may be used to connect the folds (see also FIG. 5).

In the folding stage (also called a pre-sheath stage), a balloon is folded and wrapped around a catheter. Temporary sheaths (see, e.g., 502 in FIG. 5) are then provided around the folded balloon 306. One technique used to manipulate the balloon 306 in the folding stage involves use of a folding jig, or mold. First, low pressure is applied to inflate the balloon 306. A distal end of the catheter is inserted into the mold. The balloon 306 is then pulled through the mold. The mold acts to create folds in the balloon 306.

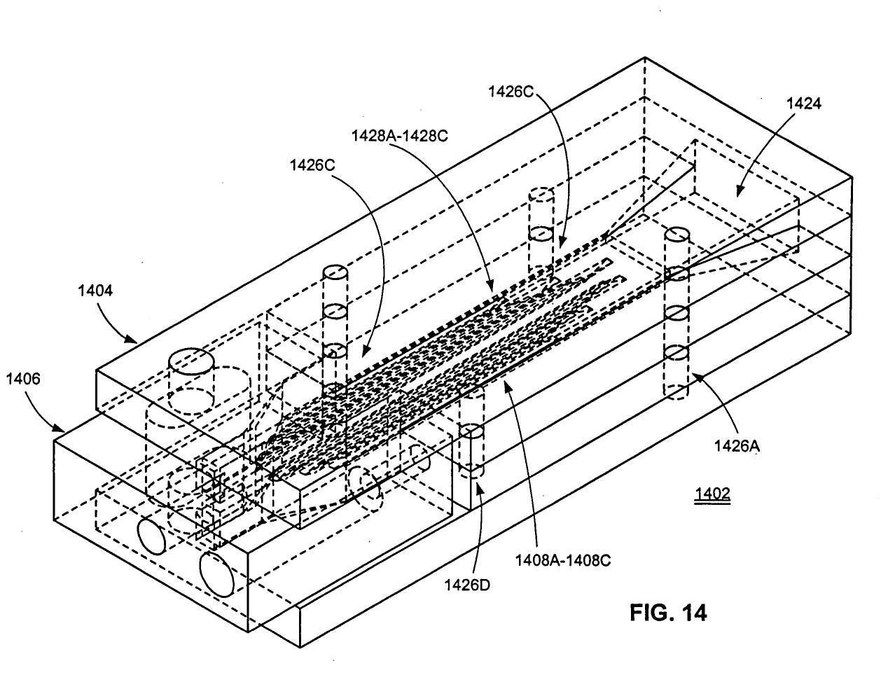

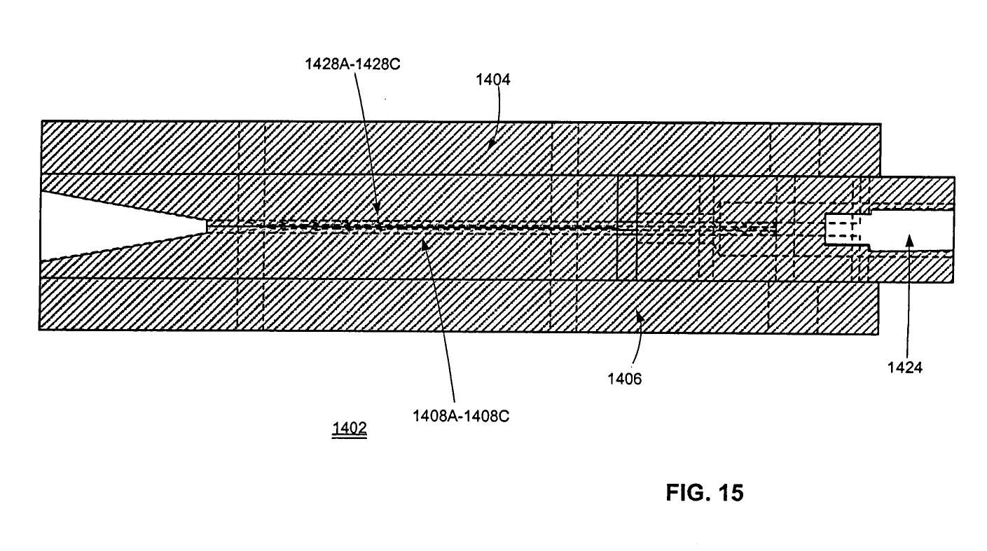

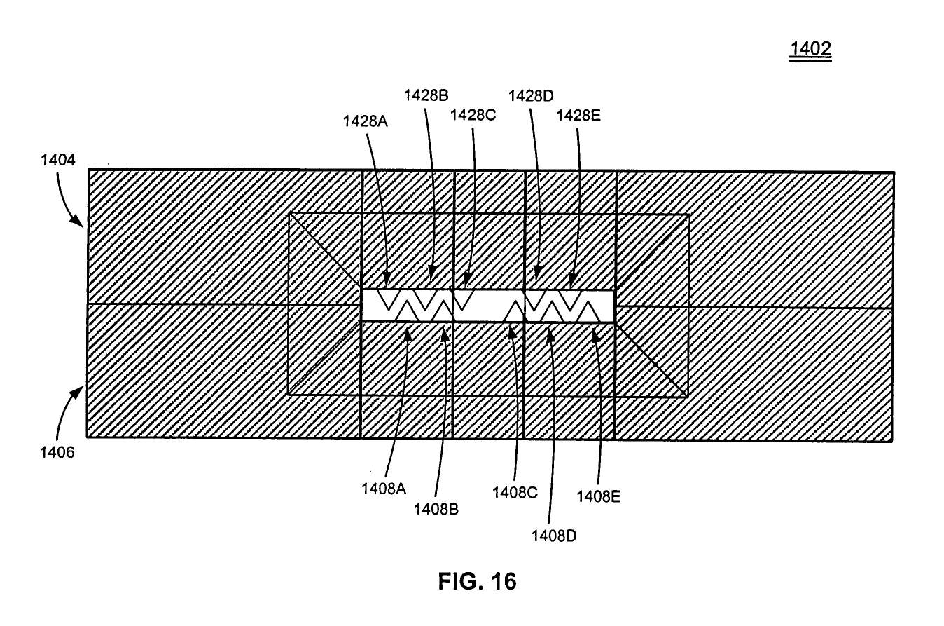

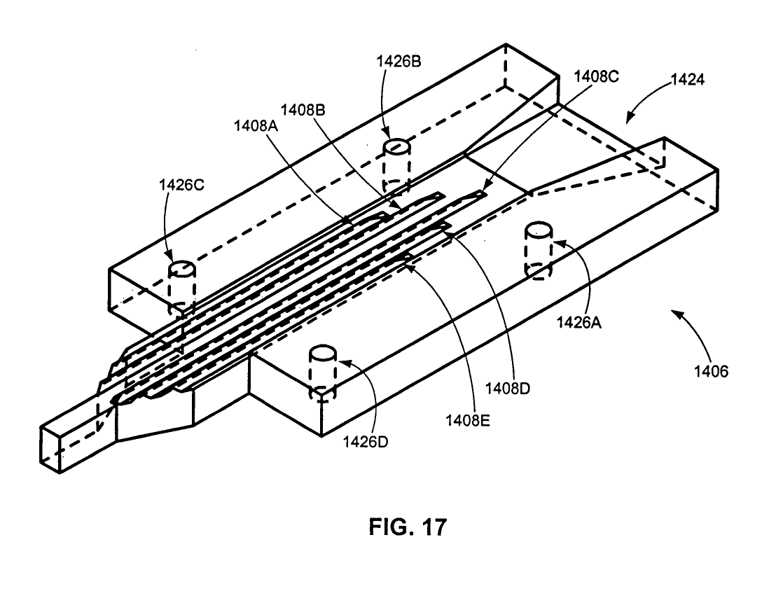

FIG. 14 illustrates an isometric view and FIG. 15 illustrates a side view of a mold 1402 used to fold the balloon 306 into the accordion arrangement. FIG. 16 illustrates a cross sectional view of upper and lower portions 1404, 1406 of the mold 1402, and FIG. 17 illustrates an isometric view of the lower portion 1406 of the mold of 1402. As shown in FIGS. 14-17, the mold 1402 includes an upper portion 1404 and a lower portion 1406. Holes 1426A, 1426B, 1426C, 1426D are used to align the upper and lower portions 1404, 1406. The balloon 306 is at least partially inflated, and then drawn through the mold 1402, thereby collapsing it and folding it.

The balloon is initially drawn from upper right towards the lower left, through an opening 1424. Ridges 1408A-1408E and 1428A-1428E are used to form the accordion structure illustrated in, e.g., FIG. 3. As the balloon 306 is drawn further through the mold 1402, the first folds that form are the innermost ones, since the first ridges encountered by the balloon 306 is, in this case, the longest central ridges, designated by 1408C and 1428C.

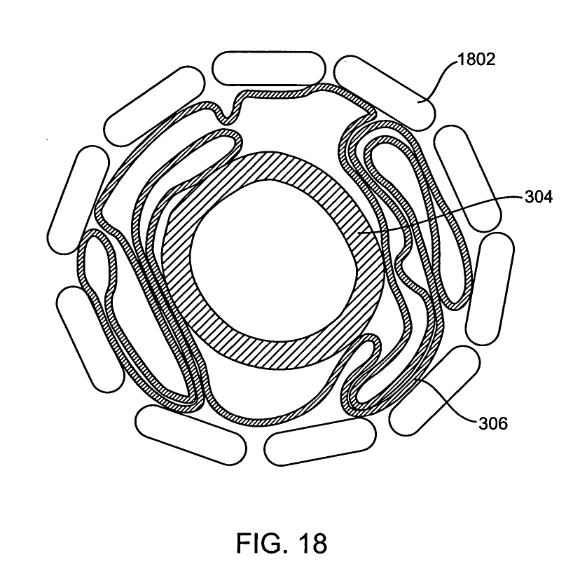

The balloon at this stage of formation, after having passed through the mold 1402, is referred to as a “folded balloon.” As the folded balloon is pulled from the mold 1402, one or more temporary guide sheaths (see 502 in FIG. 5) are placed over it to enable the mounting of the stent 1802 (see FIG. 18) on the balloon 306.

The temporary sheath 502 has an inner diameter (ID) that is sized to fit over the folded balloon 306. The temporary sheath 502 is sized to fit close to the balloon 306, but is sufficiently loose to allow room for the balloon 306 to be slightly inflated. This slight inflation or pressurization helps prevent a later-applied coating from leaking into areas between folds. The temporary sheath 502 serves as a temporary protective balloon holder.

FIG. 18 illustrates a cross sectional view of a folded balloon 306 with the stent 1802 mounted thereon. Stent 1802 is placed over the balloon 306 and one or more additional temporary guide sheaths are placed over the entire stent and balloon package. Alternatively, two temporary sheaths (called a distal sheath and a proximal sheath) may be used. Three sheaths can be used in the case of a long balloon.

Once the folded balloon 306 and stent 1802 package is placed in a temporary sheath, a positive pressure is applied to inflate the balloon 306. The remainder of the balloon 306 and stent 1802 located in the sheath are prevented from expanding by the sheath.

Heat is then applied to set the balloon folds. Typically, a balloon catheter is inserted into a heat oven and baked. Manual bake and set wrap machine methods can be used. Both the manual bake and set wrap machine methods heat the folded balloons at a sufficiently high temperature (approx. 170° F.) so that the balloon material is heat set in the folded position.

It will also be appreciated that other mold arrangements are possible and the invention is not limited to a particular arrangement of the mold 1402 shown in the figures. Also, for the tri-fold accordion (see FIG. 9), a three-piece mold (with ridges, similar to what is shown in FIGS. 14-17) may be used, with the surfaces arranged at, e.g., about 120 degrees, and pieces of the mold coming together in a manner to form the tri-fold, such as shown in FIG. 9.

The invention has a number of advantages. For example, some of the embodiments allow reduction the crossing profile, particularly in the case of a dual balloon arrangement commonly used in implantation of a bifurcated stent structure. Expansion of the stent is more uniform, since, particularly for the tri-fold arrangement, the direction of the expanding force of the balloon is radially outward (compared to the partly tangential direction in a conventional spiral-folded balloon). Similarly, the arrangement illustrated in some of the figures allows reduction of the crossing profile of a side-by-side balloon system. Additionally, the described embodiments allow expansion of the balloon, and corresponding expansion of the stent, without the balloon scraping against the stent. This is particularly important if the stent has a pharmacological coating on it.

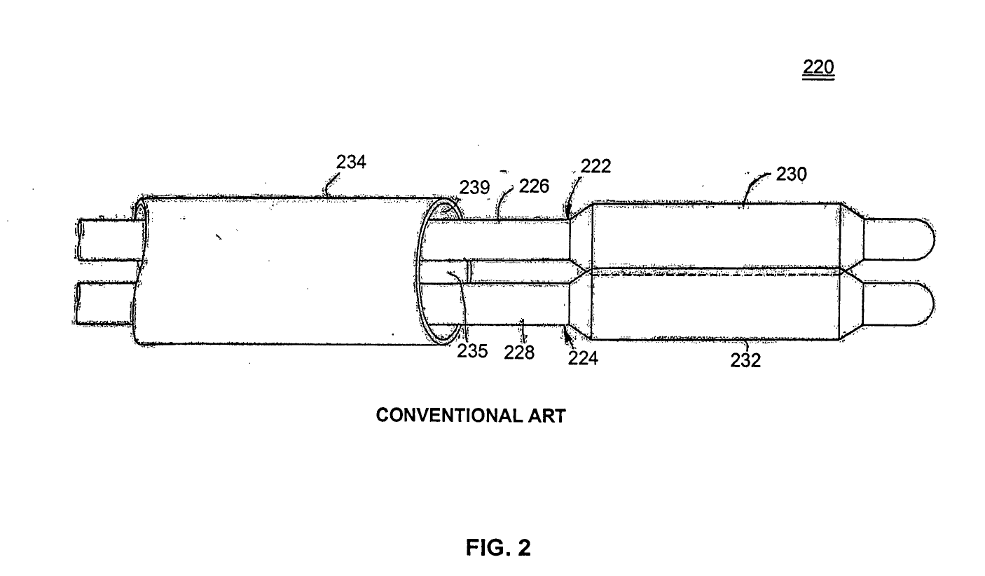

Additionally, it may be desirable to have different bending characteristics in the X and Y direction (see FIG. 7, sometimes referred to as “preferential bending”), depending on the patient anatomy. The present invention allows a preferred direction of bending for each balloon, which may be used to counteract the preferred direction of bending of a conventional two-balloon structure. In FIG. 2, it is generally easier to bend the entire structure, which consists of two inner members and two balloons, in the “out of the paper” direction as opposed to the “vertical” direction in the figure. The arrangement as shown in FIG. 7 may be used to counteract the preferential bending.

Additionally, because a structure with a roughly “rectangular” cross section has a longer circumference than a structure with a circular cross section (such as in a conventional spiral-folded balloon), it is possible to “fit more stent material” around the balloon, for the same crossing profile. Also, it is believed that stent retention is improved, compared to conventional folding arrangements.

It will be understood by those skilled in the art that various changes in form and details may be made therein without departing from the spirit and scope of the invention as defined in the appended claims. Thus, the breadth and scope of the present invention should not be limited by any of the above-described exemplary embodiments, but should be defined only in accordance with the following claims and their equivalents.

1. A balloon assembly comprising:
an inner member; and
a balloon mounted on the inner member and folded into a batwing.

2. The balloon assembly of claim 1, wherein the balloon comprises a plurality of pleats on either side of the inner member.

3. The balloon assembly of claim 2, wherein the balloon comprises an equal number of pleats on either side of the inner member.

4. The balloon assembly of claim 2, wherein the balloon is folded in an asymmetrical manner.

5. The balloon assembly of claim 1, wherein the balloon is folded in a tri-fold batwing manner.

6. The balloon assembly of claim 1, further comprising a stent mounted over the balloon.

7. The balloon assembly of claim 6, further comprising a catheter connected to the balloon.

8. The balloon assembly of claim 1, wherein the balloon is formed of any of polyethylene teraphthalate, polyethylene, polypropylene, polyvinyl chloride, and elastomer.

9. The balloon assembly of claim 1, wherein the balloon is folded into a plurality of pleats that are mirror images of each other about the inner member.

10. A dual balloon assembly comprising:
a first inner member;
a first balloon mounted on the first inner member and folded in an accordion manner;
a second inner member; and
a second balloon mounted on the second inner member.

11. The dual balloon assembly of claim 10, wherein the first balloon comprises a plurality of pleats on either side of the first inner member.

12. The dual balloon assembly of claim 11, wherein the first balloon comprises an equal number of pleats on either side of the first inner member.

13. The dual balloon assembly of claim 11, wherein the first balloon is folded in an asymmetrical manner.

14. The dual balloon assembly of claim 10, wherein the first balloon is folded in a tri-fold accordion manner.

15. The dual balloon assembly of claim 10, wherein the first balloon is folded into a plurality of pleats, each of the pleats comprising an ascending fold and a descending fold, the ascending and descending folds connected by an apex.

16. The dual balloon assembly of claim 15, wherein pleats are connected by an apex.

17. The dual balloon assembly of claim 10, wherein the first balloon is folded into a plurality of pleats, each of the pleats comprising an ascending fold and a descending fold, the ascending and descending folds connected by a curved portion.

18. The dual balloon assembly of claim 17, wherein pleats are connected by a curved portion.

19. The dual balloon assembly of claim 10, further comprising a bifurcated stent mounted over the first and second balloons.

20. The dual balloon assembly of claim 19, further comprising a catheter connected to the first and second balloons.

21. The dual balloon assembly of claim 10, wherein the first inner member is hollow.

22. The dual balloon assembly of claim 10, wherein the first balloon is formed of any of polyethylene teraphthalate, polyethylene, polypropylene, polyvinyl chloride, and elastomer.

23. The dual balloon assembly of claim 10, wherein the second balloon is folded in the accordion manner and comprises a plurality of pleats on either side of the second inner member.

24. The dual balloon assembly of claim 23, wherein the first and second balloons, in their folded state, are oriented generally perpendicular to a line connecting centers of the first and second inner members.

25. The dual balloon assembly of claim 10, wherein the second balloon is folded in the accordion manner and comprises an equal number of pleats on either side of the second inner member.

26. The dual balloon assembly of claim 10, wherein the second balloon is folded in an asymmetrical manner.

27. The dual balloon assembly of claim 10, wherein the second balloon is folded in a tri-fold accordion manner.

28. The dual balloon assembly of claim 10, wherein the second balloon is folded into a plurality of pleats, each of the pleats comprising an ascending fold and a descending fold, the ascending and descending folds connected by an apex.

29. The dual balloon assembly of claim 28, wherein adjacent pleats of the second balloon are connected by an apex.

30. The dual balloon assembly of claim 10, wherein second first balloon is folded into a plurality of pleats, each of the pleats comprising an ascending fold and a descending fold, the ascending and descending folds connected by a curved portion.

31. The dual balloon assembly of claim 30, wherein the second balloon is folded into a plurality of pleats, adjacent pleats being connected by a curved portion.

32. A balloon assembly comprising:
a plurality of inner members; and
a plurality of accordion-folded balloons mounted on corresponding inner members.

33. The balloon assembly of claim 32, wherein each balloon comprises a plurality of pleats on each side of its corresponding inner member.

34. The balloon assembly of claim 33, wherein each balloon comprises an equal number of pleats on each side of its corresponding inner member.

35. The balloon assembly of claim 32, wherein the balloons are folded into a tri-fold shape.

36. The balloon assembly of claim 32, further comprising an implantable prosthesis mounted over the accordion-folded balloons.

37. The balloon assembly of claim 36, further comprising a catheter connected to the accordion-folded balloons.

38. A method of folding a balloon comprising:
attaching a balloon to an inner member;
forming the balloon into an accordion shape; and
placing a sheath over the balloon.

39. The method of claim 38, wherein the forming step includes drawing a balloon through a mold that includes a plurality of ridges.

40. The method of claim 39, wherein the ridges guide material of the balloon to form pleats of the accordion shape.

41. The method of claim 39, further comprising the step of placing an intravascular prosthesis over the balloon.

42. The method of claim 39, wherein the forming step forms a plurality of pleats.

43. The method of claim 42, wherein the pleats are formed symmetrically about the inner member.

44. The method of claim 42, wherein the pleats are formed asymmetrically about the inner member.

45. The method of claim 38, wherein the forming step forms a tri-fold shape.

46. The method of claim 38, wherein the forming step forms a plurality of pleats of varying size.

47. A method of forming a balloon assembly comprising:
attaching a plurality of balloons to corresponding inner members;
forming each balloon into an accordion shape; and
placing a sheath over each balloon.

48. The method of claim 47, wherein the forming step includes drawing the balloons through a mold that includes a plurality of ridges.

49. The method of claim 48, wherein the ridges guide material of the balloons to form pleats of the accordion shape.

50. The method of claim 47, further comprising the step of placing an intravascular prosthesis over the balloons.

51. The method of claim 47, wherein the forming step forms a plurality of pleats.

52. The method of claim 51, wherein the pleats are formed symmetrically about each corresponding inner member.

53. The method of claim 51, wherein the pleats are formed asymmetrically about each corresponding inner member.

54. The method of claim 47, wherein the forming step forms each balloon into a tri-fold shape.

55. The method of claim 47, wherein the forming step forms a plurality of pleats of varying size.

56. A method of delivering a stent comprising the steps of:
inserting a catheter into a blood vessel, the catheter including a balloon mounted on an inner member of the catheter and a stent mounted on the balloon;
inflating the balloon to deliver the stent;
deflating the balloon; and
withdrawing the catheter from the blood vessel,
wherein the balloon mounted is folded in an accordion manner.

57. The method of claim 56, wherein the inserting step inserts a bifurcated stent.
